(19) United States
(12) Patent Application Publication
(10) Pub. No.: US 2004/0033159 A1
(43) Pub. Date: Feb. 19, 2004

(54) Activated silicon-containing-Aluminum complex for oxidizing and deodorizing purposes and method of preparation thereof

(75) Inventors: Fred Bernat, Cliffside Park, NJ (US); Siqing Song-Destro, Norwalk, CT (US)

Correspondence Address: Siqing Song-Destro, 128 Perry Avenue, Norwalk, CT, 06850, US

(73) Assignee: BB Technology Consultants, Inc., Norwalk, CT

(21) Appl. No.: 10/223,107

(22) Filed: Aug. 19, 2002

(51) Int. Cl. A01N 1/00; A61L 2/00; A61L 9/00; C23F 11/00; A61L 11/00

(52) U.S. Cl. 422/1

(57) ABSTRACT

An activated silicon-containing aluminum complex for oxidizing and deodorizing purposes, as a byproduct of the previously patent application Ser. No. 07/442,799 relating to an Explosion and Flame Proof Activated Silicon Containing Aluminum Complex. The said newly developed oxidizing and deodorizing complex contains aluminum, hydrogen, oxygen, minor amounts of halogen, and silicon. The silicon being present in amounts of at least trace and having a hexagonal structure; the ratio oxygen and hydrogen in the complex is in equal proportion, forming a peroxide group. The process of attaining such a complex comprises the steps of treating substantially pure aluminum with acid, then with mercury, then partially submerging in a halogen acid in order to create a growth of the said complex on the air-exposed part of the such treated aluminum. The growth when added to bacteria induced odor producing substances eliminates the odor. Also, having a peroxide group the said complex releases when required a sufficient amount of oxygen, in order to convert the CO in CO₂.

### BACKGROUND OF THE INVENTION

While the scientific phenomena upon which this invention is based is not fully understood, and I do not wish to be bound by any particular theory, it is believed that the sub-grain structure of aluminum appears to undergo profound changes when under chemical and electrochemical attack.

The spherical shape of the silicon trace material in aluminum changes to the hexagonal shape as a result of the “free chlorine” of the slurry, an due to the interaction of the mercury-treated aluminum with the hydrochloric acid solution. This change in structure which may be observed in the solid complex growth of this invention is also believed to be related to the phenomenon which enables the complex of this invention to release oxygen and hydrogen in presence of a catalyst.

Furthermore, when ordinary aluminum is introduced into an hydrochloric acid solution, e.g., 1N or 2N, the production of aluminum chloride (and water) takes place. However, when the mercury treated aluminum which is used in the process of this invention is placed in hydrochloric acid the behavior is quite different. While there still results the formation of aluminum chloride as well as other aluminum compounds, a complex growth on the air exposed part of the aluminum occurs. This process continues until all the aluminum is consumed.

The complex contain entrapped therein because of their clathrate properties “free chlorine” in traces (from the hydrochloric acid), oxygen, and hydrogen probably in molecular or ionic form. The silicon of the aluminum has also been changed to the hexagonal structure.

Thus the complex contains both the reaction product of aluminum and hydrochloric acid in solution, e.g. aluminum chloride, hydrogen, and hydroxide ions, and free “activated” aluminum. The aluminum contains hexagonal structured silicon as well as hydrogen and oxygen bound with.

The unusual properties of this complex may possibly also be explainable in terms of “Van der Waal's forces” of the well-know ability of particles to attract and retain on their surface different molecules, and to adhere to the particular matter. It is now capable of withholding the release of oxygen and hydrogen except in the presence of a halogen salt, such as sodium chloride, which is present in the human waste solid or liquid. The presence of the halogen salt triggers the slow and steady release of oxygen and hydrogen, until the halogen salt is completely consumed.

The principles, preferred embodiments, and modes of operation of the present invention have been described in the foregoing specification. The invention which is intended to be protected herein, however, is not to be construed as limited to the particular forms disclosed, since these are to be regarded as illustrative rather than restrictive. Variations and changes may be made by those skilled in this art without departing from the spirit of the invention.

### BRIEF SUMMARY OF THE INVENTION

The usefulness of the complex of the present invention will extend virtually to any application where the elimination of bacteria induced odors, like human or animal waste like feces and urine, applicable particularly in the cesspools, portable toilets, incontinent pads, diapers, bed mats, etc. As an additive to coal briquettes, it eliminates the probability of the development of CO as the result of inadequate aeration.

Moreover, the subject complex is non-polluting, non-toxic and safe environmentally, having no adverse impact of any kind in the atmosphere and water. It is non-corrosive and bacteria-static.

It is an object of the present invention to provide an activated silicon aluminum complex, which is capable of releasing oxygen from a hydrogen peroxide group.

Another object of the subject invention is to provide a unique complex.

Another object of the subject invention is to provide a method for the preparation of said silicon-aluminum complex.

Another object of this invention is to provide a method to apply the said complex to bacteria-induced odor producing substances, such as human and animal feces and urine.

Another object of the subject invention is to provide a method to apply the said complex to monoxide producing substances such as coal.

Still other objects will become apparent from the ensuing description and appended claims.

According to this invention, the activated aluminum complex consists essentially of aluminum and minor amounts of chlorine, activated hexagonal structured silicon, oxygen and hydrogen; the oxygen and hydrogen being bound in a peroxide group.

The complex can be prepared by the following sequence of steps:

1) Contacting aluminum metal having a purity preferably on the order of at least about 99.94% by weight, but including at least trace amount silicon, with a source of acid of a type and concentration which will remove and inhibit the formation of oxide thereon; simultaneously, or thereafter, contacting said aluminum metal with mercury or less preferably a source of mercury in an oxygen-containing atmosphere.

2) Immersing partially said mercury contacted aluminum in an acidic solution, containing halogen to effect the growth of the said complex on the oxygen-containing atmosphere exposed surface at a temperature of between ambient and not more than about 40° C.

3) Collecting the said complex-growth, storing at a temperature of between ambient, and not more than about 40° C.

4) Diluting the said complex in suitable solvents in required proportions if necessary.

5) Adding the complex to the odor producing objects either in liquid or solid form in the necessary proportion.

6) Mixing the complex with the toxic-fumes producing substances such as coal in the necessary proportion.

### BRIEF DESCRIPTION OF THE SEVERAL VIEW OF THE DRAWING

For a better understanding of the invention, reference will now be made to the accompanying drawings, wherein:

FIG. 1 is a schematic sectional elevational view of one embodiment of stage 1 of the process of the present invention.

FIG. 2 is a schematic view similar to FIG. 1, showing another optional embodiment of the stage 1 of the process of the present invention.

FIG. 3 is a schematic view similar to FIG. 1, showing the growth of the complex of the present invention in this embodiment, the aluminum is disposed substantially equidistant from the sides and bottom of the vessel.

FIG. 4 is a depiction of the structure of the untreated, inactive silicon found in non-activated form in the aluminum.

FIG. 5 is a depiction of the hexagonal structure of the silicon of the complex formed in the stages two and three of the process of the present invention.

FIG. 6 is a depiction of a soxhlet apparatus which is used in case the application of the said growth required to be in liquid form.

### DETAILED DESCRIPTION OF THE INVENTION

The activated-silicon containing aluminum complex of this invention can be conveniently prepared, utilizing a three stage process, although the process is not be narrowly construed as being limited to such. The first stage, the preparation of a form of aluminum, which can be termed “phase one”, can typically be carried out as follows:

Utilizing the apparatus of FIG. 1, an aluminum bar or rod (1) is placed, as shown, in a vessel (2), the latter constructed from any acid resistant material, but preferably of glass, and a thin layer of hydrochloric acid (3) is placed there-over slightly, covering the aluminum. In this context the shape of aluminum is not narrowly critical. However, a bar or rod shape is generally preferred. The purpose of the acid treatment is to remove the existing oxides from the aluminum surface and also to inhibit the newly creation of oxides on the aluminum surface. Hydrochloric acid is usually the acid employed for this purpose.

It is further important that the aluminum be substantially pure, in the order of, but not limited, to about 99.94% but preferably about 99.98 to 99.99% pure and also contain amounts of silicon on the order of trade to about 60 PPM to about 150 PPM. As a practical matter, whether the aluminum is sufficiently pure can be empirically determined since, if there is an abrupt rise in temperature, this indicates oxide formation and that the aluminum starting material was not sufficiently pure. Therefore, the purpose of this application, the term “substantially pure aluminum” denotes that degree of purity which is empirically determinable to be capable of being used in the process of this invention.

The aluminum is then contacted or coated with mercury or a source of mercury. As an alternative is possible to use gallium or indium for that purpose, but the mercury is preferred, placing such in a bath of the same in a similar type of apparatus, in the presence of any oxygen-containing atmosphere, such as air. The presence of oxygen in that phase of the process is imperative. In either of these preliminary steps, the temperature is not narrowly critical, but should not be such as to encourage oxide formation and/or chlorine gas release. Ambient temperature is satisfactory.

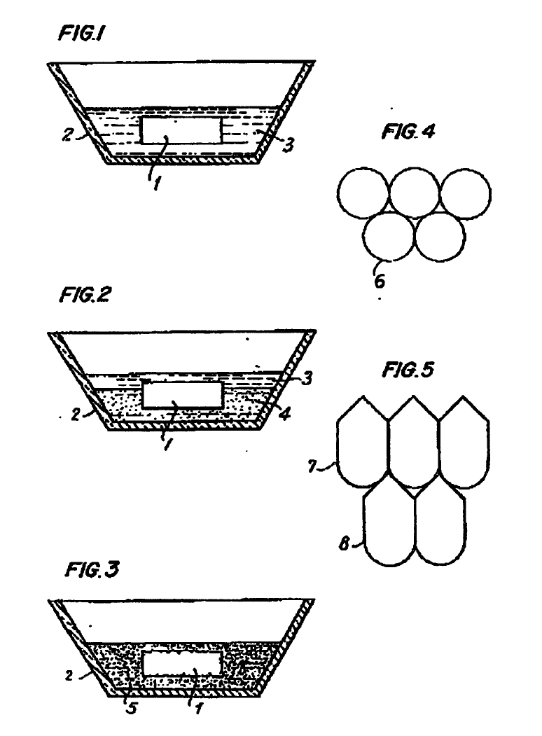

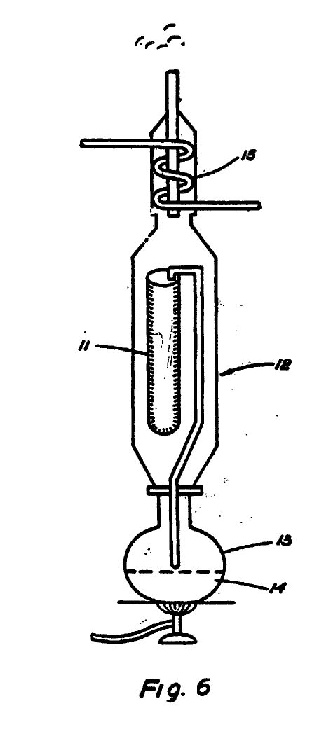

If desired, the acid and mercury contact can be made simultaneously, as shown in FIG. 2. In this figure, the aluminum (1) is immersed in the acid bath (3) and the heavier mercury bath (4), the HCL forming a layer on the bath of mercury.

Whether the apparatus on FIG. 1 or 2 or other suitable apparatus is used, the length of time of contact with the mercury can be minimal, on the order of between about fifteen and thirty seconds; longer contact however is not detrimental. Within the context of this invention, the mercury acts only as a catalyst, which effects a change in the aluminum structure. As indicate above, this changed structure is “phase two”.

The formation of “phase three” is the third stage in the process of this invention. This stage involves the formation of a complex-growth on the air exposed part of the aluminum, immersed partially in an acidic solution containing halogen. Particularly preferred among the suitable halogen solutions is hydrochloric acid.

The complex can be formed in a number of ways and the method thereof is not critical in and of itself. For example after contact with the mercury bath, the thus treated aluminum rod or bar is then partially immersed in another vessel, containing a bath of HCL. The latter should have a normality of about 1 Normal to about 2 Normal, but the actual range of concentration is empirical. When phase one, which is soluble in HCL to some extent, is partially immersed in the acid solution, a rather viscous slurry, white in color is formed. The slurry begins as a cloudy suspension and becomes increasingly dense. This is the consequence of particulate growth in and on the mercury-treated and activated aluminum rod or bar of phases one. This growth is shown in FIG. 3, wherein the thick slurry (5) is denoted as forming in the acid bath. As more and more particles form, the slurry becomes more and more viscous. Simultaneously, during the same process, a white-bluish filament like growth occurs on the oxygen containing atmosphere exposed part of the aluminum.

Depending on the size of the aluminum bar or the amount of HCL present, the formation of the slurry in the submerged part and the complex growth on the air exposed part can continue up to the entire consummation of the entire consummation of the phase one aluminum material. However as a practical matter, the reaction will usually stop before the aluminum bar is consumed completely because the slurry will become too dense, and therefore because of insufficient presence of halogen, the surface growth is inhibited. At this point, the thick slurry thus formed can be removed, partly or completely; additional HCL is then added and slurry formation on the submerged part and complex growth on the air exposed part is continued.

In the formation of the surface complex, the temperature is important, that is between ambient and not more than about 35° C. and 40° C. It should be noted that a sudden adverse rise in temperature of the reaction environment at this point could again mean that the aluminum starting material was not sufficiently pure.

Alternatively, though less desirably, the slurry and the growth can also be made “in situ” in the embodiment represented by FIG. 2. As shown in FIG. 2, the aluminum bar or rod is covered by HCL but is also partly submerged in the source of mercury. Optionally the HCL need not continue to cover the aluminum after oxide formation thereon is prevented or inhibited. A portion of the aluminum can be exposed above the surface. In either case, whether the HCL continues to cover the surface of the aluminum or not, a growth of some kind of complex occurs. This growth, itself, in this embodiment, is not the “phase two” of the slurry, but the “phase three” of this invention. In either case, the sequence has been followed of treating an oxide-free aluminum with mercury to change the structure of the aluminum and to effect its activation, and then contacting or continuing to contact said aluminum with HCL to cause the “phase two” slurry formation and the “phase three” complex growth.

In the growth complex forming step, it has been found useful, in order to avoid undesirable heat from occurring, to position the aluminum bar or rod substantially equidistant from the sides and bottom of the vessel, or at a distance from the sides and bottom of the vessel, which is essentially the same as, or greater than the diameter of the bar or rod, cylindrical rod shape being preferred. It is of course, possible to inhibit formation of undesirable heat without the above-indicated special relationship; in this event, the avoidance of oxides as a consequence of overheating would have to be constantly monitored. In this regard, for example, the treated bar could be constantly removed, re-washed, reinserted and re-coated with mercury.

While the aforesaid temperature gradients are important when forming the slurry preparatory to the subsequent formation of the complex growth, it should be noted that the slurry itself can also be formed using somewhat higher temperatures, on the order of up to about 40° C. and also starting with aluminum of slightly less purity.

### DETAILED DESCRIPTION OF THE PREFERRED EMBODIMENT

The complex of this invention is a light, amorphous, super-oxygenated material, containing aluminum, oxygen, hydrogen and at least trace amounts of unassociated chlorine and silicon. The silicon is in hexagonal structured form. The composition does not burn and it is not explosive.

By the term “activated aluminum” is meant aluminum metal which has been treated with a source of mercury, or less preferably with a source of gallium or indium, or a combination of those metals in a certain ratio. The same treated aluminum is immersed in hydrohalic acid at temperatures of between about ambient and not more than about 40° C. In this “activated” state, the aluminum metal of the slurry remains in its elemental form, but is “dathratic” in that it entraps or confines therein “free” chlorine in trace amounts. After the start of the forming of the slurry, a part of the aluminum is emerged from the halogen acid. This way the growth complex on the surface of the aluminum starts almost immediately.

The structure of the trace silicon, which is contained in the aluminum metal inherently as an impurity, is changed from its normal essentially spherical form to a hexagonal structure. This structural change may be demonstrated by an electro-microscopic examination of the complex in dry form. The change in structure of the silicon is believed to be critical, in order to attain the desired properties of the composition of the present invention. Furthermore, the clathratic entrapped free chlorine enables the present composition to release oxygen and hydrogen.

The activated-silicon containing aluminum complex of this invention may be prepared by a multi-step process. The process will be described in connection with the drawings. Utilizing the apparatus of FIG. 1, an aluminum bar or rod (1) is placed in a vessel (2) which is made preferably of glass or other acid resistant synthetic material. A thin layer of hydrohalic acid, preferably hydrochloric acid (3), is placed in vessel such that the aluminum is slightly covered. The shape of the aluminum is not critical, although a bar or rod shape is generally preferred.

The purpose of the acid treatment is to remove any oxide from the aluminum surface, and to inhibit any further development of oxide on the surface. The aluminum should be pure, generally at least about 99.9%, typically at least about 99.94%, and preferably at least about 99.99% pure. The purity of the aluminum may readily be empirically at least about 99.94%, and preferably at least about 99.99% pure. The purity of the aluminum may readily be empirically determined since the presence of oxides is demonstrated by an abrupt rise in temperature of the acid solution.

The aluminum metal should not be 100% pure, however, it is necessary that some silicon, generally from about 30 to about 100, typically from about 45 to about 95, and preferably from about 60 to about 90 parts per million (PPM) silicon be present in the aluminum. The use of less than about 30 PPM silicon will lead to a complex which will lead to a complex which will not contain sufficient hydrogen and oxygen for the desired purpose. The use of more than about 100 PPM silicon will result in process difficulties since silicon tends to heat up the solution and drive off the desired hydrogen and oxygen gases.

Other impurities may be present in the aluminum to the extent discussed above but the amount of iron present in the aluminum should generally be less than about 50, typically less than about 40, and preferably less than about 30% by weight of the silicon. It is desired to keep the iron content to a minimum because iron tends to react with, and thus deplete, the oxygen in the system.

After immersion in the acid solution, the aluminum is then contacted or coated with a source of mercury or less preferably with a source of gallium or indium, preferably by placing such in a bath of the same or similar type of apparatus. This step is carried out in the presence of an oxygen gas-containing atmosphere, such as air. The mercury acts as a catalyst which effects a change in the aluminum structure.

The temperature at which these first two steps are carried out is not critical, but should not be such as to encourage oxide and/or chlorine gas formation. A temperature range of generally from about 10 to about 45, typically from about 15 to about 35, and preferably from about 20° C. to about 30° C. may be used.

If desired, the acid and mercury contact may be made simultaneously as shown in FIG. 2, where aluminum (1) is immersed in acid bath (3) and the heavier mercury bath (4).

Regardless of weather the apparatus of FIG. 1 or 2 or other suitable apparatus is used, the length of time of contact with the mercury may be relatively short, although longer contact is not detrimental. A time of generally at least about 15, typically from about 15 to about 60, and preferably from about 30 to about 60 seconds may be employed.

The mercury acts as a catalyst, which effects a change in the aluminum structure.

The mercury-contacted aluminum thus formed is then placed in a hydrogen halide acid solution. A particularly preferred halogen acid for this purpose is hydrochloric acid. A part of the aluminum is immersed in the acidic solution, while a part of the aluminum is exposed to the oxygen-containing atmosphere. This step results in the formation of a slurry of the mercury-contacted aluminum in the acidic solution, while at the same time a complex growth starts to develop on the air exposed part of the mercury-contacted aluminum.

Alternatively, although less preferably, the slurry may be made in situ in the embodiment set forth in FIG. 2 where the aluminum bar or rod is covered by hydrochloric acid but is also partly submerged in the source of mercury. Optionally, the hydrochloric acid needs not to cover the aluminum after oxide formation thereon is inhibited. A portion of the aluminum may be exposed above the surface of the hydrochloric acid. In either case, whether the hydrogen chloride continues to cover the surface of the aluminum or not, the complex begins to grow. In either embodiment, the complex is formed by treating oxide-free aluminum with mercury to change the structure of the aluminum and to effects its activation, and then contacting (or continuing to contact) the thus treated aluminum with hydrochloric acid to cause the formation of a slurry on the submerged part of the aluminum and the formation of a growth complex on the air exposed part of the aluminum.

This complex, even in small amounts, has the unique ability of releasing oxygen and hydrogen when introduced in an oxygen and hydrogen-containing composition, such as cosmetics, medicated beauty aids, deodorants etc. Particularly is effective by using against bacteria induced odors like human and animal feces and urine. The complex is non-toxic, non-pollutant and bacteria-static. It is critical, however, for the release of oxygen and hydrogen, that a halogen salt, such as sodium chloride, be present as a catalyst.

The present invention is further illustrated by the following examples. All parts and percentages in the examples as well as in the specification and claims are by weight unless otherwise specified.

### EXAMPLE 1

Five hundred grams of aluminum metal rod, having no more than 0.02% of impurities is placed in a 36 inch long shallow glass vessel as shown by FIG. 1. The aluminum is contacted with 3N hydrochloric acid at 20° C. in amounts sufficient to cover the aluminum rod. Thereafter the aluminum rod is removed from the acid bath and immersed in a mercury bath for approximately 5 minutes under moist (relative humidity of about 40%) air-atmospheric conditions.

The mercury contacted aluminum rod is then partially reimmersed in a bath of 1N hydrochloric acid. A growth of a complex whitish-bluish in appearance started immediately. After about 72 hours all the aluminum was consumed, resulting about 1500 grams of said complex, which was removed and stored at ambient temperature.

A sample of 500 gram of commercially available fossil coal chunk was grinned to fine powder. An amount of 0.6% of said complex was mixed to the coal powder and than pressurized into a briquette. The briquette was then placed in an coal burning stove, together with a similar control sample.

Reducing the flow of the oxygen rich air to the burning process and executing periodically an analysis for CO, by a Baush and Lomb gas chromatograph, it was established that the ration of the emanated CO was 0.05 for the complex enriched sample against 0.35 for the control samples.

### EXAMPLE 2

Five grams of the growth complex in a glycol suspension had been flashed down to the tank of portable regular sized toilet. By releasing oxygen, the complex treated the sewage by reducing aerobically the liquid part to carbon dioxide and water. For the next six hours there was no odor emanation observed. Additional five grams of the complex in glycol suspension has been added to the sewage tank. For the next nine hours no odor emanation was observed.

### EXAMPLE 3

Two grams of the growth compels in powdery form has been placed between the linings of a commercially available incontinent pad. One hundred cc of human urine have been poured on. In about ten seconds all unpleasant odor emanation disappeared, which effect lasted for about eleven hours.

1. A process for preparing an activated silicon aluminum complex which is capable of releasing oxygen and hydrogen in the presence of halogen compound, said process comprising:
(a) Contacting aluminum metal having a purity of at least about 99.9% by weight and containing at least trace amounts of silicon, with a source of an acid of a type and concentration which will both remove and inhibit the formation of oxide on said metal;
(b) Contacting said acid-treated aluminum metal with a source of mercury in an oxygen-containing atmosphere;
(c) Partially immersing said mercury contacted aluminum in hydrogen halide solution for sufficient time to begin the growth of a complex on said mercury-contacted aluminum;
(d) Removing said complex from the aluminum surface and storing at ambient temperature.

2. The product of the process of claim 1.

3. The process of claim 1 wherein said aluminum is at least about 99.94% pure and contains from about 60 to about 90 PPM silicon and less than about 40% by weight iron based upon the weight of said silicon.

4. The process of claim 1 wherein step (c) is carried out at a temperature of from about 30 to about 40° C.

5. The process of claim 1 wherein said hydrogen halide acid is hydrochloric acid.

6. A process for preparing a suspension of an activated silicon aluminum complex which is capable of releasing oxygen and hydrogen in the presence of a halogen compound, said process comprising:
(a) contacting aluminum metal having a purity of at least 99.99% by weight, and containing from about 60 to about 90 PPM silicon and less than about 30% by weight iron based on the weight of said silicon, with hydrochloric acid of a concentration which will both remove and inhibit the formation of oxide on said metal;
(b) Contacting said acid-treated aluminum metal with mercury metal in an oxygen-containing atmosphere;
(c) Partially immersing said mercury contacted aluminum in a hydrogen chloride solution at temperature of from about 20° C. to about 30° C. for sufficient time to begin the growth of a complex on said mercury-contacted aluminum.

7. The product of the process of claim 6.

8. A process for releasing oxygen to the fossil fuel coal from an amount of the complex of claim 2.

9. A process for releasing oxygen to human and animal waste from an amount of the complex of claim 2.
